(19) United States
(12) Patent Application Publication
(10) Pub. No.: US 2009/0186854 A1
(43) Pub. Date: Jul. 23, 2009

(54) S-ALKYLISOTHIOURONIUM DERIVATIVES FOR THE TREATMENT OF INFLAMMATORY DISEASES

(75) Inventors: Refael Barkan, Rishon Le Zion (IL); Victor Ghicavii, Chisinau (MD)

Correspondence Address: WINSTON & STRAWN LLP;PATENT DEPARTMENT, 1700 K STREET, N.W., WASHINGTON, DC, 20006, US

(73) Assignee: Meditor Pharmaceuticals Ltd., Ness Ziona (IL)

(21) Appl. No.: 12/294,016

(22) PCT Filed: Mar. 25, 2007
(86) PCT No.: PCT/IL2007/000385

§ 371 Date: Feb. 11, 2009

Related U.S. Application Data
(60) Provisional application No. 60/784,766, filed on Mar. 23, 2006.

(51) Int. Cl. A61K 31/685

(52) U.S. Cl. 514/76

(57) ABSTRACT

The present invention relates to uses of S-alkylisothiouronium derivatives for treating inflammation. In particular, the present invention provides extended release pharmaceutical dosage forms of S-alkylisothiouronium derivatives and methods of use thereof for the treatment of various inflammatory diseases or conditions.

### FIELD OF THE INVENTION

The present invention relates to uses of S-alkylisothiouronium derivatives for treating inflammation. In particular, the present invention relates to extended release pharmaceutical dosage forms of S-alkylisothiouronium derivatives and methods of use thereof for the treatment of inflammatory diseases or conditions.

### BACKGROUND OF THE INVENTION

### Inflammation

Inflammation is characterized by the transendothelial migration of leukocytes from the vascular circulatory system into the extracellular matrix (ECM). This process is affected by a variety of cytokines, chemokines, and acute phase proteins situated within the context of the ECM.

The involvement of cytokines in the induction of acute inflammatory events, and in the transition to or persistence of chronic inflammation has been extensively studied. There is considerable evidence indicating that cytokines, such as tumor necrosis factor (TNF)-α and interleukin (IL)-1β contribute to the pathogenesis of inflammatory autoimmune diseases, specifically in rheumatoid arthritis (RA), multiple sclerosis (MS), systemic lupus erythromatosus (SLE), and in atherosclerosis.

Nitric oxide (NO) is an endogenous stimulator of the soluble guanylate cyclase. In addition to endothelium-dependent relaxation, NO is involved in a number of biological actions including cytotoxicity of phagocytic cells and cell-to-cell communication in the central nervous system.

There are at least three types of NO synthase: (i) a constitutive, Ca⁺⁺/calmodulin dependent enzyme, located in the endothelium that releases NO in response to receptor or physical stimulation; (ii) a constitutive, Ca⁺⁺/calmodulin dependent enzyme, located in the brain that releases NO in response to receptor or physical stimulation; and (iii) a Ca⁺⁺ independent enzyme which is induced after activation of vascular smooth muscle cells, macrophages, endothelial cells, and other cells by endotoxin and cytokines. Once expressed this inducible nitric oxide synthase (iNOS) generates NO continuously for long periods.

The NO released by each of the two constitutive enzymes acts as a transduction mechanism underlying several physiological responses. The NO produced by the inducible enzyme is a cytotoxic molecule for tumor cells and invading microorganisms. It also appears that adverse effects of excess NO production, in particular pathological vasodilation and tissue damage, may result largely from the NO synthesized by iNOS.

There is a growing body of evidence that NO may be involved in the degeneration of cartilage which takes place as a result of certain conditions such as arthritis and it is also known that NO synthesis is increased in rheumatoid arthritis and in osteoarthritis. US Patent application No. 20050165106 discloses amidino compounds and salts and prodrugs thereof for the treatment of a condition such as arthritis which is mediated by excessive nitric oxide production by the inducible isoform of nitric oxide synthase.

NO has an anti-inflammatory effect and inhibits the expression of many genes thought to be involved in inflammatory diseases. These include chemokines, adhesion molecules, TNF-α, interleukins, nuclear factor kappa B and cyclooxygenase-2 (COX2). However, the understanding of the interactions between NO and inflammatory markers is far from complete. Increased knowledge may open new routes to suppress inflammation.

WO 99/20251 discloses methods for decreasing or preventing non-pulmonary inflammation. The methods include causing a mammal to inhale gaseous nitric oxide. The NO gas diminishes the ability of circulating leukocytes or platelets to become activated in a manner that contributes to an inflammatory process at the site of ischemia-reperfusion or inflammation in the non-pulmonary tissue, thereby decreases or prevents non-pulmonary ischemia-reperfusion injury in the mammal.

US Patent Publication No. 20040260088 discloses novel compounds which are inhibitors of nitric oxide synthase and are thereby particularly useful in the treatment or prophylaxis of inflammatory diseases, pain and CNS diseases.

US Patent Publication No. 20030195256 discloses novel methods for the inhibition of inducible nitric oxide synthesis (iNOS) and methods of inhibiting the induction of proinflammatory cytokines.

Certain isothiourea derivatives are known in the art. For example, U.S. Pat. No. 4,490,387 teaches isothiourea derivatives which have immunosuppressant activity. U.S. Pat. No. 6,090,846 teaches substituted urea and isothiourea derivatives useful for selective inhibition of neuronal nitric oxide synthase (NOS). Those compounds are useful in treatment of cerebral ischemia, CNS trauma, pain and chronic neurodegenerative disease. U.S. Pat. No. 6,821,986 teaches isothiourea derivatives of amino acids useful for inhibiting neuronal or inducible NO synthase. US Patent Application No. 20040087653 teaches iNOS blockers including isothiourea derivatives useful for the treatment, prevention or inhibition of a respiratory disease or condition.

WO 98/13036 to the applicant of the present invention discloses the use of S-alkylisothiouronium derivatives as medicaments for increasing arterial blood pressure or for protecting subjects against hyperoxia. These compounds are suggested for the treatment of acute hypotension, e.g., shock conditions and chronic arterial hypotension or oxygen poisoning. The invention is exemplified by the hypertensive effect of S-ethylisothiouronium diethylphosphate under various conditions.

WO 02/19961 to some of the inventors of the present invention discloses the use of S-alkylisothiouronium derivatives for the prevention or treatment of headache, including migraine.

While the background art discloses uses of S-alkylisothiouronium derivatives for treating conditions that exhibit immediate effect of S-alkylisothiouronium derivatives, none of the background art discloses or suggests that extended release, sustained release or controlled release pharmaceutical dosage forms of S-alkylisothiouronium derivatives can be useful for the prevention or treatment of inflammatory diseases or conditions.

There remains an unmet need for pharmaceutical dosage forms and methods effective in treating inflammatory diseases or conditions, particularly pharmaceutical compositions and methods adapted for chronic as well as acute inflammatory diseases.

### SUMMARY OF THE INVENTION

The present invention provides extended release pharmaceutical dosage forms of S-alkylisothiouronium derivatives and methods of use thereof for the prevention and treatment of inflammatory diseases or conditions.

The present invention discloses the unexpected finding that use of extended release oral pharmaceutical dosage forms of S-alkylisothiouronium derivatives is effective in the prevention and alleviation of symptoms associated with inflammation. The extended release oral pharmaceutical dosage forms of S-alkylisothiouronium derivatives are highly advantageous as they provide a therapeutic blood concentration over an eight to twelve hour period, thus eliminating the need of frequent administration of the drug throughout the day.

It is now disclosed that extended release oral pharmaceutical dosage forms of S-alkylisothiouronium derivatives maintain the inhibitory activity of S-alkylisothiouronium derivatives on inflammation and accordingly can be useful for treating a variety of inflammatory diseases such as asthma, multiple sclerosis, and arthritis.

According to one aspect, the present invention provides an extended release pharmaceutical dosage form of an S-alkylisothiouronium derivative comprising a therapeutically effective amount of at least one compound having the general formula I:

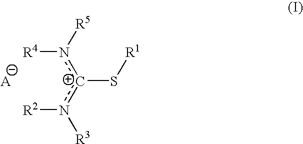

wherein,

R₁ is a linear or branched, saturated or unsaturated alkylene, comprising one to eight carbon atoms, optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;

R₂, R₃, R₄ and R₅ are each independently a hydrogen; hydroxy; an alkylene including linear or branched lower alkyl, linear or branched lower alkenyl, linear or branched lower alkynyl; lower alkoxy; alkoxyalkyl; cycloalkyl; cycloalkylalkyl; lower thioalkoxy; nitro; amino; cyano; sulfonyl; haloalkyl; carboaryloxy; carboalkylaryloxy; allyl sulfoxide; aryl sulfoxide; alkyl sulfone; aryl sulfone; alkyl sulfate; aryl sulfate; sulfonamide; thioalkyl; optionally substituted by halogen; and

A⁻ is a physiologically acceptable anion;

further comprising a polymer adapted for extended release of the compound of formula I, and optionally further comprising pharmaceutically acceptable excipients or carriers.

According to some embodiments the physiologically acceptable anion is selected from the group consisting of an anion derived from acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, glutamate, bicarbonate, p-toluenesulfonate, chloride, bromide, iodide and undecanoate.

According to other embodiments of the present invention, the physiologically acceptable anion is selected from the group consisting of an anion derived from a phosphorus containing acid, a phosphorous acid ester and a phosphorous acid amide. According to a certain embodiment, the anion is derived from a mono or di-alkyl ester of a phosphate or phosphite.

In yet other embodiments R², R³, R⁴ and R⁵ are each independently hydrogen. In some embodiments R¹ is a linear or branched alkyl.

Accordingly, in one embodiment the S-alkylisothiouronium derivative is a compound of formula (II):

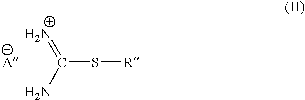

wherein

R″ is a straight or branched alkyl, optionally substituted by halogen; and

A″ (−) is an anion derived from a phosphorous containing acid.

According to some embodiments the compound is selected from the group consisting of:

S-methylisothiouronium methylphosphite;
S-methylisothiouronium dimethylphosphate;
S-ethylisothiouronium metaphosphate;
S-ethylisothiouronium ethylphosphite;
S-ethylisothiouronium diethylphosphate;
S-propylisothiouronium propylphosphite;
S-isopropylisothiouronium metaphosphate;
S-isopropylisothiouronium isopropylphosphite;
S-butylisothiouronium dibutylphosphate; and
S-isobutylisothiouronium isobutylphosphite.

In certain embodiments the compound is S-ethylisothiouronium diethylphosphate.

According to other embodiments, the polymer is selected from hydrophilic polymers, hydrophobic polymers, or a combination thereof. According to further embodiments, the hydrophilic polymer is selected from the group consisting of hydroxypropyl methylcellulose, hydroxypropyl cellulose, ethylhydroxy ethylcellulose, hydroxyethyl cellulose, carboxymethyl cellulose, sodium carboxymethyl cellulose, methyl cellulose, polyethylene oxides, polyvinyl alcohols, tragacanth, xanthan, and a mixture thereof.

According to additional embodiments, the hydrophobic polymer is selected from the group consisting of polyvinyl chloride, ethyl cellulose, polyvinyl acetate, and acrylic acid copolymers.

According to further embodiments, the extended release pharmaceutical dosage form further comprises a pharmaceutically acceptable excipient selected from the group consisting of binders, pH buffering agents, surfactants, lubricants, fillers, glidants, and antioxidants.

According to another aspect, the present invention provides a method for treating an inflammation comprising administering to a subject in need thereof a pharmaceutical composition comprising a therapeutically effective amount of at least one compound having the general formula I:

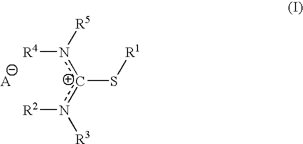

wherein,

R₁ is a linear or branched, saturated or unsaturated allylene, comprising one to eight carbon atoms, optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;

R₂, R₃, R₄ and R₅ are each independently a hydrogen; hydroxy; an alkylene including linear or branched lower alkyl, linear or branched lower alkenyl, linear or branched lower alkynyl; lower alkoxy; alkoxyalkyl; cycloalkyl; cycloalkylalkyl; lower thioalkoxy; nitro; amino; cyano; sulfonyl; haloalkyl; carboaryloxy; carboalkylaryloxy; alkyl sulfoxide; aryl sulfoxide; alkyl sulfone; aryl sulfone; alkyl sulfate; aryl sulfate; sulfonamide; thioalkyl; optionally substituted by halogen; and

A⁻ is a physiologically acceptable anion;

further comprising a pharmaceutically acceptable carrier.

According to some embodiments the physiologically acceptable anion is selected from the group consisting of an anion derived from acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, glutamate, bicarbonate, p-toluenesulfonate, chloride, bromide, iodide and undecanoate.

According to other embodiments of the present invention, the physiologically acceptable anion is selected from the group consisting of an anion derived from a phosphorus containing acid, a phosphorous acid ester and a phosphorous acid amide. According to a certain embodiment, the anion is derived from a mono or di-alkyl ester of a phosphate or phosphite.

In yet other embodiments R², R³, R⁴ and R⁵ are each independently hydrogen.

In some embodiments R¹ is a linear or branched alkyl.

Accordingly, in one embodiment the S-alkylisothiouronium derivative is a compound of formula (II):

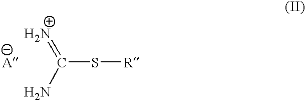

wherein

R″ is a straight or branched alkyl, optionally substituted by halogen; and

A″ (−) is an anion derived from a phosphorous containing acid.

According to some embodiments the compound is selected from the group consisting of:

S-methylisothiouronium methylphosphite;
S-methylisothiouronium dimethylphosphate;
S-ethylisothiouronium metaphosphate;
S-ethylisothiouronium ethylphosphite;
S-ethylisothiouronium diethylphosphate;
S-propylisothiouronium propylphosphite;
S-isopropylisothiouronium metaphosphate;
S-isopropylisothiouronium isopropylphosphite;
S-butylisothiouronium dibutylphosphate; and
S-isobutylisothiouronium isobutylphosphite.

In certain embodiments the compound is S-ethylisothiouronium diethylphosphate.

Routes of administration of the pharmaceutical composition of the invention include, but are not limited to parenteral, oral, rectal, vaginal, topical, pulmonary, intranasal, buccal, transdermal, ophthalmic, intravenous, intraarterial, subcutaneous, intraperitoneal, intramuscular, intraventricular, and intralesional administration routes. Preferred routes of administration include oral, topical, intramuscular and intravenous administration routes.

According to one embodiment, the subject is a mammal. According to another embodiment, the subject is a human. According to further embodiment, the subject is a non-mammalian vertebrate.

By virtue of their anti-inflammatory activity, the compounds of the present invention can be used for treating or protecting against conditions associated with inflammatory damage. Conditions associated with inflammatory damage include, but are not limited to, skin conditions including burns induced by chemical agents, thermal stimuli, or irradiation; wounds including decubitus ulcers, internal and external wounds, abscesses, and various bleedings; tissue damage including skin, hepatic, nephrologic, urologic, cardiac, pulmonary, gastrointestinal, upper airways, visual, audiologic, spleen, bone, and muscle damage; tissue transplants, graft rejection; and sepsis.

The pharmaceutical compositions comprising S-alkylisothiouronium derivatives of the present invention are useful for treating acute and chronic inflammatory diseases or conditions. Chronic Inflammatory diseases that can be treated with the compositions of the invention include, but are not limited to, hepatitis C infection, sickle cell anemia, uveitis, blepharitis, asthma, psoriasis, arthritis, inflammatory bowel disease, Crohn's disease, glomerular nephritis, autoimmune thyroiditis, systemic lupus erythematosis (SLE), multiple sclerosis, and muscle dystrophy. According to some embodiments, the chronic inflammatory disease that can be treated with the pharmaceutical composition of the invention is selected from the group consisting of asthma, SLE, and inflammatory bowel disease.

According to another aspect, the present invention provides a method for treating inflammation in a subject comprising administering to the subject in need thereof an extended release pharmaceutical dosage form of a S-alkylisothiouronium derivative, wherein the extended release pharmaceutical dosage form of the S-alkylisothiouronium derivative comprises a therapeutically effective amount of a compound of general formula I or II, a polymer adapted for extended release of the compound of formula I or II, and optionally a pharmaceutically acceptable excipient or carrier according to the principles of the present invention.

According to a currently preferred embodiment, administering the extended release pharmaceutical dosage form of the S-alkylisothiouronium derivative is performed by oral administration. According to another preferred embodiment, the extended release pharmaceutical dosage form of the S-alkylisothiouronium derivative is administered orally once or twice a day.

The present invention further provides use of a compound having a general formula (I) or (II) for the manufacture of a medicament for the treatment of inflammatory diseases or conditions. Preferably, the medicament is formulated in an extended release pharmaceutical dosage form according to the principles of the present invention.

These and other embodiments of the present invention will become apparent in conjunction with the description and claims that follow.

### DETAILED DESCRIPTION OF THE INVENTION

The present invention for the first time discloses the finding that S-alkylisothiouronium derivatives are effective in preventing and alleviating the symptoms associated with inflammation.

The present invention relates to the use S-alkylisothiouronium derivatives including, but not limited to, S-ethylisothiouronium diethylphosphate, for the treatment of inflammatory diseases or conditions.

S-alkylisothiouronium derivatives have been disclosed in PCT patent application publications WO 98/13036 and WO 02/19961, incorporated by reference herein in their entirety. WO 98/13036 teaches certain S-alkylisothiouronium derivatives for the treatment of diseases or disorders associated with hypotension and hyperoxia. WO 02/19961 teaches S-alkylisothiouronium derivatives useful in treating headache and in particular migraine.

### DEFINITIONS

As used herein, the terms “inflammation”, “inflammatory response”, or the like, generally refer to a biological process which involves the immune system. Inflammation is the first response of the immune system to infection, injury or irritation in a body. Though inflammation is an important component of innate immunity, if left unabated, it may result in severe and sometimes irreparable tissue damage. Inflammation also contributes to the pathophsiology of numerous disorders such as, for example, tissue reperfusion injury following myocardial infarction, system lupus erythematosis, inflammatory bowel disease including Crohn's disease and ulcerative colitis, asthma, atherosclerosis, arthritis including rheumatoid arthritis and osteoarthritis, ankylosing spondylitis, psoriasis, chondrocalcinosis, gout, blepharitis and cachexia.

An inflammatory response may include bringing leukocytes and plasma molecules to sites of infection or tissue injury. Inflammation may generally be characterized as causing a tissue to have one or more of the following characteristics: redness, heat, swelling, pain and dysfunction of the organs involved. At the tissue level, the principle effects of an inflammatory response may include increased vascular permeability, recruitment of leukocytes and other inflammatory cells to the site of the inflammatory response, changes in smooth muscle contraction and the synthesis and release of proinflammatory mediator molecules, including eicosanoids.

The terms “compound having anti-inflammatory activity” and “anti-inflammatory compound” refer to compounds that inhibit or arrest inflammatory processes and are used interchangeably throughout the specification.

As used herein, the term “subject” refers to a vertebrate such as a mammal, including both human and other mammals. The methods of the present invention are preferably applied to human subjects.

As used herein the term “therapeutically effective amount” or “therapeutically efficient” as to a drug dosage, refer to dosage of the compounds of the invention that when administered to a subject is capable of exerting anti-inflammatory activity. The “therapeutically effective amount” may vary according, for example, the physical condition of the subject, the age of the subject and the severity of the inflammatory disease, disorder or condition.

As used herein, the term “alkylene” refers to a saturated or unsaturated hydrocarbon chain including straight chain or branched chain alkyl, alkenyl or alkynyl.

As used herein, the term “alkyl” refers to a saturated hydrocarbon chain containing 1 to 30, preferably 1 to 6 carbon atoms, such as, but not limited to, methyl, ethyl, n-propyl, isopropyl, n-butyl, isobutyl, tert-butyl, n-pentyl, n-hexyl, and the like. As used herein the term allyl also reads on haloalkyls, which contain halogen atoms. Alkyl also includes heteroalkyl with heteroatoms of sulfur, oxygen and nitrogen.

“Alkenyl” and “alkynyl” are used to mean straight or branched chain hydrocarbon groups having from 2 to 12 carbons and unsaturated by a double or triple bond respectively, such as vinyl, allyl, propargyl, 1-methylvinyl, but-1-enyl, but-2-enyl, but-2-ynyl, 1 methylbut-2-enyl, pent-1-enyl, pent-3-enyl, 3-methylbut-1-ynyl, 1,1-dimethylallyl, hex-2-enyl and 1-methyl-1-ethylallyl.

The term “cycloalkyl” is used herein to mean cyclic radicals, including but not limited to, cyclopropyl, cyclopentyl, cyclohexyl, and the like.

The term “cycloalkylalkyl” as used herein refers to a cycloalkyl group appended to a lower alkyl radical, including, but not limited to cyclohexylmethyl.

The “alkoxyalkyl” mentioned for the R substituents is preferably a group containing a total of 1 to about 22 carbon atoms. For example, methoxyethyl, methoxypropyl, methoxybutyl, ethoxyethyl, ethoxypropyl, ethoxybutyl, n-propoxyethyl, and iso-propoxyethyl, are suitable.

The term “alkoxy” as used herein refers to an alkyl group attached to the parent molecular group through an oxygen atom.

The term “halo” or “halogen” as used herein refers to I, Br, Cl or F.

The term “carboxy” as used herein refers to the radical —COOH.

The term “ester” refers to —COOR; wherein R is linear or branched alkyl.

The term “amide” refers to —CONH₂, —CONHR or —CONR₂ wherein R is independently selected from hydrogen and straight or branched alkyl.

The term “cyano” as used herein refers to the radical —CN.

The term “carboaryloxy” refers to compounds including carbophenoxy.

The term “alkyl sulfoxide” refers to residues of the formula (—SOR) where R is a straight chain or branched alkyl.

As used herein a “pharmaceutical composition” refers to a preparation of one or more of the compounds described herein or physiologically acceptable salts or prodrugs thereof, with other chemical components such as pharmaceutically acceptable carriers and excipients. The purpose of a pharmaceutical composition is to facilitate administration of a compound to an organism.

The term “pharmaceutically acceptable” means suitable for administration to a subject, e.g., a human. For example, the term “pharmaceutically acceptable” can mean approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned.

As used herein, reference to any of the terms “disease”, “condition” and “disorder” is to be taken as a reference to all three terms. Diseases, conditions and disorders that may be specifically mentioned are: osteoarthritis, rheumatoid arthritis, rheumatoid spondylitis, gouty arthritis and other arthritic conditions, inflamed joints; eczema, psoriasis, dermatitis or other inflammatory skin conditions such as sunburn; inflammatory eye conditions including uveitis, glaucoma and conjunctivitis; lung disorders in which inflammation is involved, for example, asthma, bronchitis, chronic obstructive pulmonary disease, pigeon fancier's disease, farmer's lung, acute respiratory distress syndrome; bacteraemia, endotoxaemia (septic shock), aphthous ulcers, gingivitis, pyresis, pain, meningitis and pancreatitis; conditions of the gastrointestinal tract including inflammatory bowel disease, Crohn's disease, atrophic gastritis, gastritis varialoforme, ulcerative colitis, coeliac disease, regional ileitis, peptic ulceration, irritable bowel syndrome, reflux oesophagitis, damage to the: gastrointestinal tract resulting from infections by, for example, Helicobacter pylori, or from treatments with non-steroidal anti-inflammatory drugs; and other conditions associated with inflammation.

The term “extended release pharmaceutical dosage form” as used herein refers to a dosage form which, due to the special technology of preparation, provides maintenance of a therapeutic blood level of the active ingredient for 8 hours up to 24 hours. The term “sustained release pharmaceutical dosage form” and “controlled release pharmaceutical dosage form” are used interchangeably throughout the application and refer to dosage forms that maintain a therapeutic blood level of the S-alkylisothiourea derivatives of the invention for extended period of time, namely 8 hours or more.

The present invention provides a method of treating inflammatory diseases or conditions by at least one S-alkylisothiouronium derivative.

According to currently preferred embodiments, the inflammatory diseases are selected from the group consisting of asthma, blepharitis, uveitis, and cachexia. According to other currently preferred embodiments, the inflammatory diseases are selected from the group consisting of multiple sclerosis, inflammatory bowel disease, and arthritis.

### Asthma

Asthma affects about 150 million people worldwide and is the most prevalent chronic disease in childhood. High prevalence of childhood asthma observed during the last decades predicts the growing prevalence of asthma in the near future unless appropriate preventive measures are undertaken. Asthma affects about 10 million Americans, about a third of whom are under 18 years of age. In the United States alone billions of dollars are spent annually on asthma-related health care. The episodic breathing difficulty that characterizes asthma is brought about by a combination of three primary factors including 1) bronchospasm, that is to say, variable and reversible airway obstruction due to airway muscle contraction, 2) inflammation of the airway lining, and 3) bronchial hyper-responsiveness that results in excessive mucus in the airways. Triggers of asthma attacks vary among individuals, but include allergens such as dust mites and mold, environmental pollutants, viral agents, and physical exertion or exercise.

Asthma includes atopic asthma; non-atopic asthma; allergic asthma; bronchial asthma; essential asthma; true asthma; intrinsic asthma caused by pathiophysiologic disturbances; extrinsic asthma caused by environmental factors; emphysematous asthma; exercise-induced asthma; occupational asthma; infective asthma caused by bacterial, fungal, protozoal, or viral infection; non-allergic asthma; incipient asthma; wheezy infant syndrome; and any asthma of unknown etiology, or pathogenesis.

### Eye Inflammation

Examples of eye disorders associated with an inflammation include, but are not limited to, bacterial conjunctivitis, fungal conjunctivitis, viral conjunctivitis, uveitis, blepharitis, keratic precipitates, macular edema, and inflammation response after intra-ocular lens implantation.

Uveitis is a general term referring to inflammation of the uveal tract (iris, ciliary body, and choroid). Although it refers primarily to inflammation of this vascular structure, adjacent structures such as retina, vitreous, sclera, and cornea are also frequently involved. Patients most afflicted are 20-50 years of age, with a marked decrease after the age of 70.

Anterior uveitis (iritis) occurs more frequently than posterior uveitis and affects the iris and/or ciliary body. Anterior uveitis, especially acute uveitis, is usually marked by eye pain, redness, photophobia (light sensitivity), mildly decreased vision, and tearing, and may be unilateral or bilateral depending on the etiology. The critical sign of uveitis is cells and flare (white blood cells and protein leakage) in the anterior chamber. Many cases of acute, non-recurrent, anterior uveitis tend to be idiopathic and are treated primarily with anti-inflammatory/steroid drops. Other causes of acute anterior uveitis (which can be recurrent) include: ocular trauma, post-surgical inflammations, medications, contact lens-related complications, HLA-B27 antigen, and inflammatory/autoimmune conditions (ankylosing spondylitis, inflammatory bowel disease, Reiter's syndrome, etc.) In cases of chronic anterior uveitis, the etiology is usually due to other systemic conditions such as juvenile rheumatoid arthritis, sarcoidosis, herpes simplex/herpes zoster/varicella, tuberculosis, and Fuch's heterochromia iridocyclitis.

Posterior uveitis involves the posterior segment of the eye (with corresponding retinal/choroidal inflammation and lesions). The onset may be acute but most often is insidious with little pain and minimum photophobia and blurred vision. Diseases with associated posterior uveitis include Lyme disease, toxoplasmosis, toxocariasis, histoplasmosis, and syphilis.

Various types of palliative treatments have been used to treat this condition. The most common treatment includes the administration of eye drops, which contain emollients and other ingredients designed to ease the discomfort due to the inflammation and to eliminate the redness associated with the condition. These treatments, however, have not been entirely satisfactory.

For example, current treatments often involve frequent applications of a medicinal eye drop. Unfortunately, however, many commercially available eye drops include preservatives, an ingredient that may be quite harmful to the eye. As such, frequent application of the commercially available eye drops may not be healthy for the eye.

Blepharitis is a chronic inflammation of the eyelids and is one of the most common disorders of the eye. It usually results from a dysfunction of the small oil glands of the eyelids. When these glands are not secreting the oils involved in tear formation, blepharitis can result.

As a part of the ageing process, the skin of the eyelid can stretch, the muscles weaken and fatty deposits accumulate around their eyelids. When this happens, oils and other secretions may build up on the eyelid surface and eyelashes, resulting in a crusting and flaking material that can enter the eye. Swelling or inflammation of the eyelids is also common. Other symptoms can include a sandy, gritty sensation, with burning, itching and/or redness.

Although blepharitis is a chronic problem, applying warm compresses and eyelid scrubs can help control it. In some cases, it may be necessary to prescribe an antibiotic ointment or pills. A person can also undergo blepharoplasty, a corrective eyelid surgery that helps to remove excess tissue and fatty deposits around the eyes. Typically, however, ophthalmologists simply use either their hands or Q-tips to press against the eyelids and excrete the oils that cause blepharitis.

### Detection of the Anti-Inflammatory Activity

The anti-inflammatory activity of the compounds of the invention can be detected by measuring the inhibitory effect of the compounds on T cell function in in vitro assays including, but not limited to, inhibition of T cell adhesion, inhibition of T cell migration, inhibition of TNFα secretion by T cells and monocytes, and inhibition of TNFα and IFNγ secretion by activated T cells.

The inhibitory activity of the compounds of the invention may be monitored in in vivo inflammatory conditions such as, for example, in delayed type hypersensitivity (DTH), adoptive DTH, experimental autoimmune encephalomyelitis (EAE), adjuvant arthritis (AA), colitis, and Con A-induced hepatic injury. Other methods of determining the inhibition of T cell function include detection of neutrophil migration into inflammatory sites in vivo. Typically, inflammation sites can be created by placing polyvinyl sponges at various subcutaneous sites (see for example Price et al., (1987) J. Immunol. 139: 4114-4177). The sponges are placed subcutaneously by making sterile incisions. Blood samples are drawn from the subcutaneous sponges at various times before and after administration of the compounds of the invention, the number of neutrophils is determined and the reduction in neutrophil number indicates the efficacy of the treatment.

Thus, according to the invention a therapeutically effective amount of the compounds of the present invention is the amount that reduces or inhibit T cell activity by at least 10 percent, more preferably by at least 50 percent, and most preferably by at least 90 percent, when measured in an in vitro assay. Preferably, the compounds of the present invention are useful for inhibiting inflammatory disease in a subject in need thereof. Thus, a therapeutically effective amount of the compounds of the invention is the amount that when administered to a subject is sufficient to inhibit, preferably to eradicate, inflammation.

### Preferred Embodiments of the Present Invention

Without excluding other options, which are listed below, presently S-ethylisothiouronium diethylphosphate is the preferred compound for preventing and/or treating inflammatory diseases, disorders or conditions.

According to one aspect of the present invention there is provided an anti-inflammatory medicament for patients comprising, as an active ingredient, a compound having the general formula (I):

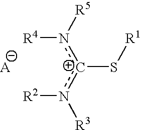

wherein,

R₁ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;

R₂, R₃, R₄ and R₅ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen; and

A⁻ is a physiologically acceptable anion.

Preferably, the physiologically acceptable anion is derived, without limitation, from a phosphorus containing acid, the group consisting of an anion derived from a phosphorus containing acid, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, hydrochloride, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, phosphate, glutamate, bicarbonate, p-toluenesulfonate, chloride, bromide, iodide and undecanoate.

According to currently preferred embodiments of the invention described below, the physiologically acceptable anion is an anion derived from a phosphorus containing acid, more preferably the anion derived from a phosphorus acid ester or amide, most preferably the anion is derived from a mono or di-alkyl ester of a phosphorous containing acid.

In some embodiments each of the R², R³, R⁴ and R⁵ substituents are H. In certain embodiments R¹ is a straight or branched alkyl. In one embodiment, R¹ is a C₁-C₆ alky.

Accordingly, in certain embodiments the S-alkylisothiouronium derivative is a compound of formula (II):

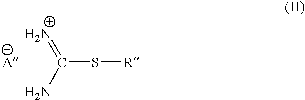

wherein

R″ is a straight or branched alkyl, optionally substituted by halogen; and

A″ (−) is an anion derived from a phosphorous containing acid.

Preferred examples of S-alkylisothiouronium derivatives according to formula (II) which can be used to prevent and/or treat inflammatory diseases or conditions, include, but are not limited to, S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutylisothiouronium isobutylphosphite.

The present invention provides a pharmaceutical composition comprising as an active ingredient a compound of formula (I) or (II) and a pharmaceutically acceptable carrier or diluent.

These compounds are known to be safe for human use, as it is well known in the art that phosphorus containing derivatives of S-alkylisothiouronium have a low toxicity and their LD₅₀ (lethal dose 50%) is in the range of 100-1000 mg/kg, which is far above the therapeutic doses of these compounds.

Toxicological studies indicated that the compounds of the invention are not toxic when administered as either a single or repeated dose. For example, the LD₅₀ for S-ethylisothiouronium diethylphosphate is up to 400 mg/kg in rats, values 300-400 fold higher than the therapeutically recommended dose of 0.6-1.2 mg/kg.

Based on animal toxicological studies and on the accumulated data on human patients, S-ethylisothiouronium diethylphosphate was approved for a Phase I clinical trial. In this study, the pharmacokinetic profile as well as safety of escalating doses (0.6-2.4 mg/kg) of S-ethylisothiouronium diethylphosphate were assessed in 12 healthy male subjects. The results of the Phase I study indicated that S-ethylisothiouronium diethylphosphate was well tolerated in doses up to 1.2 mg/kg with no recorded adverse events.

### Methods of the Invention and Administration Routes

In another aspect of the present invention there is provided a method for treating inflammatory diseases or conditions. The method according to this aspect of the present invention is performed by administering to a subject a therapeutically effective amount of a pharmaceutical composition comprising as an active ingredient a compound having the general formula (I):

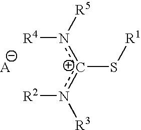

wherein

R₁ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;

R₂, R₃, R₄ and R₅ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen;

A⁻ is a physiologically acceptable anion.

Any suitable route of administration may be employed for providing a patient with an effective amount of an S-alkyl isothiouronium derivative. Suitable routes of administration include, for example, oral, topical, rectal, nasal, buccal, parenteral (such as, intravenous, intrathecal, subcutaneous, intramuscular, intrasternal, intrahepatic, intralesional, intracranial, intra-articular, and intra-synovial), transdermal (such as, for example, patches), and the like.

A compound according to the present invention can be administered to a treated subject per se, or in a pharmaceutical composition where it is mixed with suitable carriers or excipients. Furthermore, the compound can be administered as monotherapy or as combination therapy.

Pharmaceutical compositions may also include one or more additional active ingredients, such as, but not limited to, conventional anti-inflammatory drugs.

Anti-inflammatory drugs that can be administered in combination with the compound of the present invention include but are not limited to: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminun; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

Combination therapies can involve the administration of the active ingredients as a single dosage form or as multiple dosage forms administered at the same time or at different times.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer.

An example of a currently preferred injectable formulation is presented in Table 1.

TABLE 1
Exemplary injectable formulation of the present invention

| | Quantity | |
|---|---|---|
| Material | per ml | Function |
| S-ethylisothiouronium | 100 mg | Active ingredient |
| diethylphosphate | | |
| Monosodium Phosphate | 1.59 mg | Excipient, pH 5.0-6.0 |
| | | buffer component |
| Disodium Phosphate | 0.33 mg | Excipient, pH 5.0-6.0 |
| 7H₂O | | buffer component |
| Water for Injection | To make up | Excipient, Solvent |
| (WFI) | 1.00 ml | |

For transmucosal administration, penetrants appropriate to the barrier to be permeated are used in the formulation. Such penetrants for example DMSO or polyethylene glycol are generally known in the art.

For oral administration, the compounds can be formulated readily by combining the active compounds with pharmaceutically acceptable carriers well known in the art.

An example of a currently preferred oral formulation is presented in Table 2.

TABLE 2
Exemplary oral formulation of the present invention

| Material | Quantity per tablet | |
|---|---|---|
| S-ethylisothiouronium | 50 | mg |
| diethylphosphate | | |
| Lactose | 101 | mg |
| Colloidal Silicone Dioxide | 1.0 | mg |
| Microcrystalline cellulose | 40 | mg |
| Crosspovidone (PVP) | 4.0 | mg |
| Stearic acid | 4.0 | mg |

| | |
|---|---|
| Coating materials | Up to 5% of the weight of |
| | the compressed tablet core |

The carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The pharmaceutical compositions of the present invention can be formulated in an extended release pharmaceutical dosage form as known in the art (see, for example, U.S. Pat. Nos. 6,605,303; 6,419,958; 6,245,357; and Krowczynski, L. in Extended-Release Dosage Forms, CRC Press, Inc. 1987, the content of which is incorporated by reference as if fully set forth herein).

Thus, an extended release pharmaceutical dosage form of the S-alkylisothiouronium derivatives of the present invention comprise an S-alkylisothiouronium derivative, a polymer, and optionally one or more additional pharmaceutically acceptable excipient or carrier.

Polymers that can be used for the preparation of the extended release pharmaceutical dosage form of the present invention include hydrophilic polymers, hydrophobic polymers, and a combination thereof.

Suitable hydrophilic polymers are for instance hydroxypropyl methylcellulose, hydroxypropyl cellulose, ethylhydroxy ethylcellulose, hydroxyethyl cellulose, carboxymethyl cellulose, sodium carboxymethyl cellulose, methyl cellulose, polyethylene oxides, polyvinyl alcohols, tragacanth, and xanthan. These polymers can be used alone or in mixtures with each other.

Hydrophobic polymers are exemplified by for instance polyvinyl chloride, ethyl cellulose, polyvinyl acetate and acrylic acid copolymers, such as Eudragith™. The polymers can be used alone or as mixtures. Alternatively or additionally, hydrophobizing agents can be used for the hydrophobic matrix such as for instance cetanol, cetostearyl alcohol, cetyl palmitate, waxes lice carnauba wax, paraffin, magnesium stearate, sodium stearyl fumarate, and medium- or long-chain glycerol esters alone or in any mixtures.

The extended release pharmaceutical dosage forms of the invention can further comprises binders such as for instance sugars, polyvinyl pyrrolidine, starches and gelatin; surfactants such as non-ionic surfactants such as for instance polysorbate 80, or ionic surfactants such as for instance sodium lauryl sulfate; lubricants such as for instance magnesium stearate, sodium stearyl fumarate, or cetyl palmitate; fillers such as for instance sodium aluminum silicate, lactose, or calcium phosphate; glidants such as for instance talc and aerosol; and antioxidants.

A currently exemplary embodiment of the extended release pharmaceutical dosage form of the present invention is S-ethylisothiouronium diethylphosphate in admixture with microcrystalline cellulose and hydroxypropyl methylcellulose. Formed as beads or spheroids, the drug containing formulation is coated with a mixture of ethyl cellulose and hydroxypropylmethyl cellulose to provide the desired level of coating.

For buccal administration, the compositions may take the form of tablets or lozenges formulated in conventional manner.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Pharmaceutical compositions for parenteral administration include aqueous solutions of the active preparation in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

Alternatively, the active ingredient may be in powder form for constitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited o, calcium carbonate, calcium phosphate, various sugars, starches, cellulose derivatives, gelatin and polymers such as polyethylene glycols.

It may be desirable to administer the pharmaceutical composition of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material. Administration can also be by direct injection e.g., via a syringe, at the site of an inflammation.

For topical application, an anti-inflammatory compound of the invention can be combined with a pharmaceutically acceptable carrier so that an effective dosage is delivered, based on the desired activity. Accordingly, an anti-inflammatory compound of the invention can be applied to the skin for treating wounds and skin diseases such as, for example, psoriasis. The carrier may be in the form of, for example, and not by way of limitation, an ointment, cream, gel, paste, foam, aerosol, suppository, pad or gelled stick

For directed internal topical applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents.

An anti-inflammatory compound of the invention can be delivered in a controlled release system. For example, the anti-inflammatory compound can be administered in combination with a biodegradable, biocompatible polymeric implant, which releases the compound over a controlled period of time at a selected site. Examples of preferred polymeric materials include polyanhydrides, polyolthoesters, polyglycolic acid, polylactic acid, polyethylene vinyl acetate, copolymers and blends thereof (see Medical applications of controlled release, Langer and Wise (eds.), 1974, CRC Pres., Boca Raton, Fla.). A controlled release system can be placed in proximity of the therapeutic target, thus requiring only a fraction of a systemic dose.

According to some embodiments, the present invention provides compositions for treating an eye inflammatory disorder. For ophthalmic administration, the composition can be formulated readily by combining the compound of the invention with pharmaceutically acceptable carriers well known in the art. Such carriers enable the compounds of the invention to be formulated as liquids, gels, emulsions, thermogels, slurries, suspensions, and the like, for ophthalmic use by a patient. Alternatively the composition can be formulated as a solid, for resuspension. In other embodiments, pharmaceutical compositions for ophthalmic administration include aqueous solutions of the active ingredients in water-soluble form.

The mode of administration of the present compositions depends on the form of the composition. For example, if the composition is a solution, drops of the composition may be applied to the eye, e.g., from a conventional eyedropper. In general, the present compositions may be applied to the surface of the eye in substantially the same way as conventional ophthalmic compositions are applied.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate inflammatory diseases or condition in the subject being treated.

Determination of a therapeutically effective amount is well within the capability of those skilled in the art, especially in light of the detailed disclosure provided herein.

The exact formulation, route of administration and dosage can be chosen by the individual physician in view of the patient's condition. (See e.g., Fingl, et al., 1975, in “The Pharmacological Basis of Therapeutics”, Ch. 1 p. 1).

The amount of a composition to be administered will, of course, be dependent on the subject being treated, the severity of the inflammatory diseases or conditions, the manner of administration, the judgment of the prescribing physician, etc. For example, doses up to 1.2 mg/kg of the compounds of the invention would be well tolerated in healthy volunteers and represents a therapeutic alternative for the treatment of patients.

### Uses of the Compounds

The present invention relates to a method of treating or protecting against an inflammation by administering to a subject in need thereof a therapeutically effective amount of a pharmaceutical composition comprising a compound having anti-inflammatory activity according to the principles of the invention and a pharmaceutically acceptable carrier.

As anti-inflammatory agents, the compounds of the invention are expected to be efficacious in all diseases, disorders, or conditions that involve inflammation or inflammatory activity.

According to some embodiments, the subject to be treated or protected with the pharmaceutical compositions comprising the compounds of the invention is a mammal. According to other embodiments, the mammal is a human. Treatment or protection against inflammation may be accomplished in the fetus, newborn, child, adolescent as well as in adults and old persons, whether the inflammation to be treated is spontaneous, chronic, of traumatic etiology, a congenital defect or a teratogenic phenomenon.

According to some embodiments, the inflammation is associated with an inflammatory disease, disorder or condition.

According to other embodiments, the inflammatory disease is selected from the group consisting of chronic inflammatory disease and acute inflammatory disease.

According to additional embodiments, the inflammation to be treated by the pharmaceutical compositions of the invention is associated with hypersensitivity.

According to further embodiments, the hypersensitivity is selected from the group consisting of immediate hypersensitivity, antibody mediated hypersensitivity, immune complex mediated hypersensitivity, T lymphocyte mediated hypersensitivity and delayed type hypersensitivity.

According to further embodiments, the inflammation to be treated by the pharmaceutical compositions of the invention is associated with autoimmune disease.

According to some embodiments, the inflammation to be treated by the pharmaceutical compositions of the invention is associated with an infectious disease. According to additional embodiments, the infectious disease is selected from the group consisting of chronic infectious disease, subacute infectious disease, acute infectious disease, viral disease, bacterial disease, protozoan disease, parasitic disease, fungal disease, mycoplasma disease and prion disease.

According to further embodiments, the inflammation to be treated is associated with a disease associated with transplantation of a graft. The disease associated with transplantation of a graft is selected from the group consisting of graft rejection, chronic graft rejection, subacute graft rejection, hyperacute graft rejection, acute graft rejection and graft versus host disease. According to additional embodiments, the graft is selected from the group consisting of a cellular graft, a tissue graft, an organ graft and an appendage graft.

According to other embodiments, the inflammation to be treated is associated with an allergic disease. According to some embodiments, the allergic disease is selected from the group consisting of asthma, hives, urticaria, pollen allergy, dust mite allergy, venom allergy, cosmetics allergy, latex allergy, chemical allergy, drug allergy, insect bite allergy, animal dander allergy, plant allergy and food allergy.

According to further embodiments, the inflammation to be treated by the pharmaceutical compositions of the invention is associated with a tumor. According to additional embodiments, the tumor is selected from the group consisting of a malignant tumor, a benign tumor, a solid tumor, a metastatic tumor and a non-solid tumor.

According to another embodiment, the inflammation is associated with septic shock.

According to further embodiment, the inflammation to be treated is associated with anaphylactic shock.

According to yet further embodiment, the inflammation to be treated is associated with toxic shock syndrome.

According to additional embodiments, the inflammation to be treated is associated with a prosthetic implant. According to some embodiments, the prosthetic implant is selected from the group consisting of a breast implant, a silicone implant, a dental implant, a penile implant, a cardiac implant, an artificial joint, a bone fracture repair device, a bone replacement implant, a drug delivery implant, a catheter, a pacemaker and a respirator tube.

According to other embodiments, the inflammation to be treated is associated with an injury. According to some embodiments, the injury is selected from the group consisting of an abrasion, a bruise, a cut, a puncture wound, a laceration, an impact wound, a concussion, a contusion, a thermal burn, frostbite, a chemical burn, a sunburn, a desiccation, a radiation burn, a radioactivity burn, smoke inhalation, a torn muscle, a pulled muscle, a torn tendon, a pulled tendon, a pulled ligament, a torn ligament, a torn cartilage, a bone fracture, a pinched nerve and a gunshot wound.

According to further embodiments, the inflammation to be treated is a musculo-skeletal inflammation. According to some embodiments, the musculo-skeletal inflammation is selected from the group consisting of arthritis, muscle inflammation, myositis, a tendon inflammation, tendinitis, a ligament inflammation, a cartilage inflammation, a joint inflammation, a synovial inflammation, carpal tunnel syndrome and a bone inflammation.

A pharmaceutical composition of the invention is administered in a manner compatible with the dosage formulation, and in a therapeutically effective amount. The quantity to be administered depends on the subject to be treated, capacity of the subject's blood hemostatic system to utilize the active ingredient, and the degree of inflammation required to be eradicated. Precise amounts of active ingredient required to be administered depend on the judgment of the practitioner and are peculiar to each individual.

The methods of treatment according to the present invention include both therapeutic and prophylactic utility. Thus, the pharmaceutical compositions comprising the compounds of the invention can be administered prior to the occurrence of an inflammation or can be administered after the inflammation has appeared.

According to some embodiments, repeated application may enhance the anti-inflammatory activity of the compounds of the invention and may be required in some applications. Additionally, the compounds of the invention can be administered alone or in conjunction with other therapeutic modalities. Thus, it is appropriate to administer the compounds of the invention as part of a treatment regimen involving other therapies, such as surgery and/or drug therapy.

Having now generally described the invention, the same will be more readily understood through reference to the following examples, which are provided by way of illustration and are not intended to be limiting of the present invention.

### EXAMPLES

The following assays are used to demonstrate the effect of S-alkylisothiouronium derivatives on inflammatory responses in vitro and in vivo.

### Example 1

### Anti-Inflammatory Properties of S-Alkylisothiouronium Derivatives

Specific aspects of the inflammatory response cascade are mediated by cytokines, such as TNF-α, IFN-γ, IL-2 and IL-1β and by inflammatory mediators such as COX-2 and PGE₂. Reducing the levels of these pro-inflammatory agents is an important step in reducing inflammation. Activated cells of the immune system also produce these agents, and the purpose of this study is to test the impact of the S-alkylisothiouronium derivatives on secretion of these inflammatory agents from activated macrophages and T cells. The levels of secretion in the various test groups are measured by ELISA and the level of inhibition by S-alkylisothiouronium derivatives is calculated versus the vehicle treated group.

### Quantitation of Protein Using ELISA.

The technique used to quantify the amount of a given protein in a liquid sample, either tissue culture supernatant or body fluid, is based on Enzyme Linked ImmunoSorbent Assay (ELISA) methodology. Either commercially available or established in house, the assay is based on the capture of the protein of interest by specific antibodies bound to the bottom of an ELISA plate well. Unbound material is washed away; the captured protein is then exposed to a secondary antibody generally labeled with horseradish peroxidase (HRP) or alkaline phosphatase (ALP). Again the unbound material is washed away, the samples are then incubated with the appropriate substrate yielding a calorimetric reaction. The reaction is stopped and reading is performed in a spectrophotometer at the appropriate wavelength. Samples are tested at least in duplicate and the appropriate standard curve, consisting of serial dilutions of a known protein, is incorporated on each plate. Concentration of the protein in the sample is calculated from the standard curve.

### Macrophage Activation.

RAW 264.7 macrophages, a mouse cell line (ATCC # TIB-71), are grown in Dulbecco's modified Eagle's medium (DMEM) with 4 mM L-glutamine adjusted to contain 1.5 g/L sodium bicarbonate, 4.5 g/L glucose, and 10% heat inactivated fetal bovine serum. Cells are grown in tissue culture flasks and seeded at appropriate density into 24 well tissue culture plates. 0.5×10⁶ Raw cells in one milliliter are stimulated with 2 μg/ml Lipopolysaccharide (LPS) E. coli 055:B5 (DIFCO Laboratories). The mouse macrophages are pre-treated for one hour with controls or 10 μM of the S-alkylisothiouronium derivatives and later on activated with LPS. Dexamethasone is used as a positive control at 50 nM. Supernatant is collected 4 hours (for PGE₂) and 24 hours (for IL-1β and TNF-α) after activation, and the levels of the inflammatory cytokines and mediators under study are determined by ELISA, as previously described. Inhibition by S-alkylisothiouronium derivatives is calculated versus vehicle treated cells.

### T Cell Activation.

Jurkat cells (human acute lymphoma T-cell line; ATCC # TIB-152) are grown in RPMI 1640 medium with 2 mM L-glutamine adjusted to contain 1.5 g/L sodium bicarbonate, 4.5 g/L glucose, 10 mM HEPES, 1.0 mM sodium pyruvate, and 10% heat inactivated fetal bovine serum. Cells are grown in tissue culture flasks and seeded at appropriate density into 24 well tissue culture plates. 2×10⁶ cells in one milliliter are stimulated using 10 ng/ml of PMA (Sigma) and 1 μM A23187 calcium ionophore (Sigma). Cyclosporin A (Sandoz), a known immunosuppressive drug, is used as positive control. The controls and the S-alkylisothiouronium derivatives are added at indicated concentrations one hour before stimulation. Supernatant is collected 24 hours after stimulation and the levels of the inflammatory cytokines and mediators under study are measured by ELISA, as previously described. Inhibition by the S-alkylisothiouronium derivatives is calculated versus vehicle treated cells.

### Mast Cell Activation.

Mast cells are multifunctional bone marrow derived cells that upon activation release potent inflammatory mediators. Release is detected either from preformed granules, through the process of degranulation, or following stimulation-induced de novo synthesis. The molecules released by mast cells include biogenic amines such as histamine, chemokines, cytokines, enzymes, growth factors, peptides, arachidonic acid products and proteoglycans. It should be noted that mast cells are also known to play a key role in generating pain signal. RBL-2H3 cells (rat basophilic leukemia cell line; ATCC # CRL-2256) provide a model for testing mast cell activation.

RBL-2H3 cells are grown in EMEM medium with Earle's BSS, 2 mM L-glutamine adjusted to contain 1.5 g/L sodium bicarbonate, 0.1 mM non essential amino acids, 1.0 mM sodium pyruvate, and 15% heat inactivated fetal calf serum. Cells are grown in tissue culture flasks and seeded at appropriate density into 24 well tissue culture plates. 2×10⁵ cells in one milliliter are stimulated by either one of the following. First, the IgE dependent method wherein after overnight plating, medium is replaced and cells are sensitized for one hour with medium containing 0.5 μg/ml anti-DNP (dinitrophenyl) conjugated to IgE (Sigma, Cat. No. D-8406). Cells are then washed twice with PBS and exposed to fresh pre-warmed medium containing 0.1 μg/ml DNP-HSA (dinitrophenyl-human serum albumin, Sigma, Cat. No. A-6661). S-alkylisothiouronium derivatives and controls, diluted in DMSO, are added before the ultimate stimulus at final concentration not exceeding 0.1% DMSO. The degranulation process is allowed to proceed at 37° C. for various periods of time, depending on the mediator to be assessed, and 200 μl of supernatant are then collected. For instance, cells are stimulated for 1 hour for monitoring histamine secretion, and for three hours for monitoring serotonin, TNF-α and IL-4 secretion.

Alternatively, the stimulation of the mast cells is achieved using 10 ng/ml of PMA (Sigma) and 1 μM A23187 calcium ionophore (Sigma). The Src family inhibitor PP1 or the PKC inhibitor GF109203X (both from Calbiochem) are used as positive control. The controls and S-alkylisothiouronium derivatives are added at indicated concentrations before stimulation. Supernatant is collected up to 24 hours after stimulation, depending upon the mediator under study, and the levels of this agent are measured in an ELISA as previously described. Inhibition is calculated versus vehicle treated cells.

### Example 2

### Treatment of Inflammation: the Ear Edema Model in the Mouse

The anti-inflammatory activity of the S-alkylisothiouronium derivatives is tested in vivo using an ear edema model in mice. This test system utilizes various inflammation inducers, including Croton oil (CO) and Arachidonic acid (AA) with the outcome assessed by measuring ear tissue swelling. Nonsteroidal anti-inflammatory drugs have been shown to reduce swelling in this model (Young, J. M. et al., J. Invest. Dermatol. 82: 367-71, 1984). The ability of the S-alkylisothiouronium derivatives to prevent or diminish the inflammatory response to these stimulants is indicative of their systemic anti-inflammatory capability.

S-alkylisothiouronium derivatives are dissolved in appropriate diluent and injected i.p. in adult male ICR mice (30 g average body weight, Harlan, Israel) after dilution with sterile 0.9% sodium chloride to desired final concentrations according to required doses. Various doses of S-alkylisothiouronium derivatives are checked ranging from 0 to 30 mg/kg. Each treatment group is composed of 8-10 animals while the vehicle treated group is composed of 16 animals. Inflammation is immediately induced by applying 20 μl of 50% CO in acetone to the outer surface of one ear, the contralateral ear is exposed to acetone only and served as control. Ear thickness is determined (in 0.01 mm units) 3 hours after CO application using a dial thickness gauge. Finally the ears are trimmed, an ear punch of 6 mm diameter is removed and weighed. The ear edema is expressed as the ratio of ear punch weight of the CO treated ear versus the contralateral Acetone treated ear. Results are calculated as % inhibition as compared to vehicle treated animals.

### Example 3

### Treatment of Inflammation: the Paw Edema Model in the Mouse

The purpose of this study is to test in vivo the anti-inflammatory activity of the S-alkylisothiouronium derivatives in paw edema induced by injection of 1% carrageenan in the animal hind paw. Female Balb/c mice (20 g average body weight, Harlan, Israel) are anesthetized with a combination of xylazine and pentobarbitone diluted in sterile saline, 15 and 6 mg/kg i.p. respectively. Anesthetized mice are injected subcutaneously, in the subplantar region of one (right) paw with 0.05 ml of 1% w/v Carrageenan in sterile water. The contralateral (left) paw is not injected, as data from the literature, showed that injection of 0.05 ml of normal saline did not affect later thickness or volume measurements. The S-alkylisothiouronium derivatives, or known anti-inflammatory controls, are dissolved in vehicle and further diluted 1:20 or 1:50 in sterile saline prior to i.p. injection that takes place immediately before the carrageenan injection. Three hours after injection the animals are resedated following the previously described procedure. Paw thickness is measured using a dial thickness gauge (Spring-dial, constant low pressure gauge, Mitutoyo, TG/L-1, 0.01 mm) and paw volume is measured using a plethysmometer. Paw Edema is expressed as the difference between the right treated and the left untreated paws of the same animal, either as Δ Paw Volume (ΔPV) in millimeters cube or as Δ Paw Thickness (ΔPT) in millimeters. Each group comprises at least 10 animals. Results can be further normalized to the ΔPV and ΔPT values of each treatment group at 0 mg/kg (vehicle only). At the end of the study, animals are euthanized with an i.p. injection of 100 mg/kg pentobarbitone.

The results are first calculated as ΔPV or ΔPT, and then further analyzed as % inhibition by comparing the effect of S-alkylisothiouronium derivatives treatment versus vehicle on paw volume or thickness. The differences among various treatment groups are analyzed by analysis of variance (ANOVA) followed by post-hoc Fisher test. A value of p<0.05 is considered to be statistically significant.

When results are expressed as % inhibition of paw thickness, normalized to vehicle, and plotted against the dose of the test compound, the resulting pattern is an initial slope up to a maximal observed effect (MOE) at a given dose followed by a plateau at higher doses.

### Example 4

### Treatment of Inflammatory Pain: the Paw Edema Model in Rats

The purpose of this study is to test the anti-inflammatory pain activity of the S-alkylisothiouronium derivatives in paw edema induced by injection of 2% λ carrageenan in the animal hind paw. Male Sprague Dawley rats (200 g average body weight, Harlan, Israel) are transiently sedated by placement on dry ice for the duration of the injections. Rats are injected subcutaneously, in the subplantar region of one (right) paw with 0.1 ml of 2% w/v λ Carrageenan in sterile saline. The contralateral (left) paw is not injected, as data from the literature, showed that injection of 0.1 ml of normal saline did not affect later analgesic measurements. The test compounds, including known anti-inflammatory controls, are dissolved in vehicle and further diluted 1:20 or 1:50 in sterile saline prior to i.p. injection that takes place immediately after the carrageenan injection. Before induction of inflammatory pain and three hours after injection, the animal reactions to pain stimuli were tested in two systems. The first stimulus was thermal and assessed by the Plantar Test according to Hargreaves, using Ugo Basile Model 7370. The scale is set to an intensity of 50 arbitrary units. The latency time till the animal lift a paw as a reaction to the thermal stimulus is recorded for both the inflamed and non-inflamed hind paws. The second stimulus is mechanical (tactile) and assessed using a Dynamic Plantar Sesthesiomether (Ugo Basile Model 73400-002). The system is set on maximal force of 50 grams and the force applied is gradually increased at the rate of 10 g/sec. At the end of the study, animals are euthanized with an i.p. injection of 100 mg/kg pentobarbitone.

The results are measured as the differences between the two hind paws at time 0 and 3 hours both as ΔLT, for the latency time in the thermal part of the study, and as ΔForce, for the mechanical part of the study. Results are expressed as mean±SE for each treatment group and the differences among those groups are analyzed by analysis of variance (ANOVA) followed by post-hoc Tukey's test. A value of p<0.05 is considered to be statistically significant.

The anti inflammatory pain activity of the S-alkylisothiouronium derivatives is compared not only to an opiate but also to non-steroidal anti-inflammatory drugs (NSAID). Three drugs are tested in this model: Celecoxib (COX-2 inhibitor), Ketoprofen (COX-1 inhibitor) and Diclofenac (COX-1 and COX-2 inhibitor). The NSAIDs are tested at three doses: 5, 10 and 20 mg/kg and the intermediate dose of 10 mg/kg is selected for the rest of the study.

### Example 5

### Effect of the S-Alkylisothiouronium Derivatives in a Model for Inflammatory Bowel Disease

The anti-inflammatory activity of S-alkylisothiouronium derivatives compounds is tested in the study of acetic acid-induced IBD in rats. Male Sprague Dawley rats (10 weeks old, 200-250 g, Harlan, Israel) are lightly anaesthetized by i.p. injection of a ketamine: rompun combination (100:10 mg/kg respectively). A polyethylene catheter (outer diameter 1.7 mm) is inserted through the rectum 5 cm into the colon, and 2 ml of 5% acetic acid are then slowly administered into the colon. Fifteen seconds later the colon is washed with 3 ml saline and 15 seconds later with additional 3 ml of saline. Immediately after, each group of animals is treated with S-alkylisothiouronium derivatives or controls. All treatments are administered once daily for 7 days. Animals are clinically followed for 1 week. During this period, the following parameters are daily monitored and recorded: body weight, presence of blood in the stool and stool consistency.

The clinical outcome is analyzed using analysis of variance (ANOVA) followed by Duncan's post-hoc test. A non-parametric test (Wilcoxon Rank Sum Test) is used for evaluating the gross pathology findings.

### Example 6

### Effect of the S-Alkylisothiouronium Derivatives in a Model for Multiple Sclerosis

Experimental autoimmune encephalitis in female mice is a well known model for multiple sclerosis. Female C57BL mice are intracardially injected with a compound of the invention or the vehicle NaCl 0.9% (volume of injection-0.25 ml/animal) under light pentobarbital (15 mg/kg) anesthesia Immediately thereafter myelin oligodendritic glycoprotein (MOG) 35-55, emulsified with Complete Freund's Adjuvant, is subcutaneously administered into 4 sites on the back, adjacent to each of the forelimbs and hindlimbs, each injection is performed at volume of 50 μl. Each animal is i.p. injected with pertusis toxin in PBS (200 ng/mouse). The pertusis toxin injection is repeated after 2 days. The animals are evaluated for neurological score from 0 (no effect) to 4 (severe neurological symptoms including paralysis).

### Example 7

### Effect of the S-Alkylisothiouronium Derivatives on Pulmonary Functions

Mice (Balb/c, female, 8 weeks old) are first sensitized by ip injection of the antigen (Ag) ovalbumin. Thereafter, the mice are challenged 4 times by aerosol inhalation of the same Ag dispersed by nebulizer in a plexiglass chamber for about 20 min at one-week intervals. On day 28, the mice are first treated with an aerosol of the tested compound dissolved in 0.1 M NaHCO₃. As a result, the aerosol is inhaled by the animals for about 10 to 20 minutes. After inhalation, the animals are immediately challenged by inhalation (as above) of the sensitizing antigen (5% OVA in PBS) for 20 min. At the end of this treatment, the mice are immediately tested for pulmonary functions in conscious, freely moving state using plethysmography.

The degree of bronchial constriction is monitored by the enhanced pause and its relation to airway resistance, impedance and intrapleural pressure in the mouse. Bronchoalveolar lavage (BAL) samples are obtained from these mice by cannulating the trachea, injecting 0.8 ml ice-cold saline (×2) and subsequently aspirating the BAL fluid. Following these tests, mice are sacrificed using general anesthesia with brevital (1 mg/ml). Their chests wall are opened, blood withdrawn and lungs are perfused with cold PBS and examined for cytology and histology.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1-32. (canceled)

33. An extended release pharmaceutical dosage form comprising a therapeutically effective amount of a compound having the general formula: 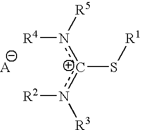
a polymer in an amount sufficient to delay the release of the compound from the formulation when it is administered to a subject, and optionally a pharmaceutically acceptable excipient or carrier, wherein:
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S; and
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen.

34. The extended release pharmaceutical dosage form of claim 33, wherein the physiologically acceptable anion is selected from the group consisting of an anion derived from a phosphorus containing acid, a phosphorous containing acid ester, a phosphorous containing acid amide, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, phosphate, glutamate, bicarbonate, p-toluenesulfonate, chloride, bromide, iodide and undecanoate.

35. The extended release pharmaceutical dosage form of claim 33, wherein the physiologically acceptable anion is an anion derived from a phosphorus containing compound and the excipient is present and is selected from the group consisting of binders, surfactants, lubricants, fillers, glidants, and antioxidants.

36. The extended release pharmaceutical dosage form of claim 33, wherein the physiologically acceptable anion is a phosphorus containing acid selected from the group consisting of a mono-alkyl ester of a phosphorus containing acid and di-alkyl ester of a phosphorus containing acid.

37. The extended release pharmaceutical dosage form of claim 33, wherein R¹ is selected from the group consisting of a linear alkyl and a branched alkyl and R², R³, R⁴ and R⁵ are each independently hydrogen.

38. The extended release pharmaceutical dosage form of claim 33, wherein the compound is of formula: 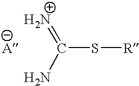
wherein
R″ is a straight or branched alkyl, optionally substituted by halogen; and
A″ is an anion derived from a phosphorous containing acid.

39. The extended release pharmaceutical dosage form of claim 38, wherein the compound is selected from the group consisting of S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutylisothiouronium isobutylphosphite.

40. The extended release pharmaceutical dosage form of claim 39, wherein the compound is S-ethylisothiouronium diethylphosphate.

41. The extended release pharmaceutical dosage form of claim 33, wherein the polymer is a hydrophilic polymer, a hydrophobic polymer, or a combination of hydrophilic and hydrophobic polymers, and wherein the hydrophilic polymer is selected from the group consisting of hydroxypropyl methylcellulose, hydroxypropyl cellulose, ethylhydroxy ethylcellulose, hydroxyethyl cellulose, carboxymethyl cellulose, sodium carboxymethyl cellulose, methyl cellulose, polyethylene oxides, polyvinyl alcohols, tragacanth, and xanthan and the hydrophobic polymer is selected from the group consisting of polyvinyl chloride, ethyl cellulose, polyvinyl acetate and acrylic acid copolymers; such that the dosage form provides a therapeutic blood concentration over an 8 to 12 hour period after administration to a subject.

42. A method for treating inflammation which comprises administering to a subject in need of such treatment a therapeutically effective amount of a pharmaceutical composition comprising as an active ingredient a compound having the general formula: 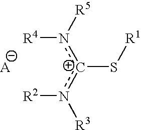
and a pharmaceutically acceptable carrier, wherein:
R¹ is a linear or branched saturated or unsaturated alkylene, comprising one to eight carbon atoms optionally substituted with one or more substituent selected from the group consisting of halogen, primary, secondary, tertiary or quaternary amine, primary, secondary or tertiary alcohol, or interrupted by one or more heteroatom selected from the group consisting of O, N, and S;
R², R³, R⁴ and R⁵ are each independently a hydrogen, hydroxy, linear or branched lower alkyl, linear or branched lower alkenyl, linear or branched lower alkynyl, lower alkoxy, alkoxyalkyl, cycloalkyl, cycloalkylalkyl, lower thioalkoxy, nitro, amino, cyano, sulfonyl, haloalkyl, carboaryloxy, carboalkylaryloxy, alkyl sulfoxide, aryl sulfoxide, alkyl sulfone, aryl sulfone, alkyl sulfate, aryl sulfate, sulfonamide, thioalkyl, optionally substituted by halogen; and
A is a physiologically acceptable anion.

43. The method of claim 42, wherein the physiologically acceptable anion A is selected from the group consisting of an anion derived from a phosphorus containing acid, a phosphorous containing acid ester, a phosphorous containing acid amide, acetate, adipate, alginate, citrate, aspartate, benzoate, benzenesulfonate, bitartarate, bisulfate, butyrate, camphorate, camphorsulfonate, digluconate, glycerophosphate, hemisulfate, heptanoate, hexanoate, fumarate, 2-hydroxyethanesulfonate, isothionate, lactate, maleate, methanesulfonate, nicotinate, 2-naphthalenesulfonate, oxalate, palmoate, pectinate, 3-phenylpropionate, pivalate, propionate, succinate, tartrate, thiocyanate, phosphate, glutamate, bicarbonate, p-toluenesulfonate, chloride, bromide, iodide and undecanoate.

44. The method of claim 43, wherein the physiologically acceptable anion A is an anion derived from a phosphorus containing selected from the group consisting of a mono-alkyl ester of a phosphorus containing acid and di-alkyl ester of a phosphorus containing acid.

45. The method of claim 42, wherein R¹ is selected from the group consisting of a linear alkyl and a branched alkyl and R², R³, R⁴ and R⁵ are each independently hydrogen.

46. The method of claim 43, wherein the compound is of formula II: 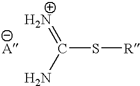
wherein R″ is a straight or branched alkyl, optionally substituted by halogen.

47. The method of claim 46, wherein the compound is selected from the group consisting of S-methylisothiouronium methylphosphite; S-methylisothiouronium dimethylphosphate; S-ethylisothiouronium metaphosphate; S-ethylisothiouronium ethylphosphite; S-ethylisothiouronium diethylphosphate; S-propylisothiouronium propylphosphite; S-isopropylisothiouronium metaphosphate; S-isopropylisothiouronium isopropylphosphite; S-butylisothiouronium dibutylphosphate; and S-isobutylisothiouronium isobutylphosphite.

48. The method of claim 46, wherein the compound is S-ethylisothiouronium diethylphosphate.

49. The method of claim 42, wherein the pharmaceutical composition is formulated in an extended release pharmaceutical dosage form that provides a therapeutic blood concentration over an 8 to 12 hour period after administration to a subject and is administered via intraperitoneal, parenteral, intravenous, oral, topical or transdermal administration route.

50. The method of claim 42, wherein the subject is a mammal and the inflammation is associated with an inflammatory disease, disorder or condition.

51. The method of claim 42, wherein the mammal is a human and the inflammatory disease is selected from the group consisting of chronic inflammatory disease and acute inflammatory disease.

52. The method of claim 51, wherein the inflammatory disease is selected from the group consisting of rheumatoid arthritis, asthma, inflammatory bowel disease, psoriasis, Crohn's disease, ulcerative colitis, multiple sclerosis, blepharitis, uveitis, glomerular nephritis, delayed type hypersensitivity, autoimmune thyroiditis, lupus erythematosus, tissue transplants, graft rejection, atherosclerosis, chronic alcoholic liver disease, non-alcoholic steatohepatitis, and chronic pancreatitis.
